(19) United States
(12) Patent Application Publication
(10) Pub. No.: US 2024/0066284 A1
(43) Pub. Date: Feb. 29, 2024

(54) ANTIMICROBIAL ELECTROCHEMICAL FABRIC AND METHOD FOR MANUFACTURING THE SAME

(71) Applicant: CATCHER TECHNOLOGY CO., LTD., TAINAN CITY (TW)

(72) Inventors: Chih-hsing Lin, TAINAN CITY (TW); YU-YU CHO, TAINAN CITY (TW); CHAO-LUN HSIAO, TAINAN CITY (TW)

(21) Appl. No.: 18/455,576

(22) Filed: Aug. 24, 2023

Related U.S. Application Data
(60) Provisional application No. 63/401,128, filed on Aug. 26, 2022.

(51) Int. Cl. A61N 1/04; D01D 5/00; D01F 1/10; D01F 11/00

(52) U.S. Cl. CPC A61N 1/0468; A61N 1/0476; D01D 5/003; D01F 1/103; D01F 11/00; D10B 2509/022

(57) ABSTRACT

An antimicrobial electrochemical fabric and a method for manufacturing the same are provided. The method for manufacturing the antimicrobial electrochemical fabric includes the following steps: providing an electro-spinning polymer solution, in which the electro-spinning polymer solution includes a polymer and a plurality of antimicrobial metal precursors; electro-spinning the electro-spinning polymer solution into a polymer fiber for formation of a sheet structure, in which the plurality of antimicrobial metal precursors are distributed on the polymer fiber; and reducing the plurality of antimicrobial metal precursors into a plurality of antimicrobial metal particles, so as to form the sheet structure into the antimicrobial electrochemical fabric.

### CROSS-REFERENCE TO RELATED PATENT APPLICATION

This application claims the benefit of priority to the U.S. Provisional Patent Application Ser. No. 63/401,128, filed on Aug. 26, 2022, which application is incorporated herein by reference in its entirety.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a fabric and a method for manufacturing the same, and more particularly to an antimicrobial electrochemical fabric and a method for manufacturing the same.

### BACKGROUND OF THE DISCLOSURE

With the rapid development of medical technology, a wet compress therapy (also referred to as a wet wrap therapy) has been widely recognized. Studies have shown that a moist and closed environment for wounds can promote the release of growth factors, stimulate cell proliferation, accelerate epidermal cell migration, enhance white blood cell function, and promote microvascular regeneration.

Wound healing is a dynamic biological process that includes four continuous, overlapping, and precisely programmed phases: hemostasis (from 0 to several hours after injury), inflammation (from 1 to 3 days), proliferation (from 4 to 21 days), and remodeling (from 21 days to 1 year). For an adult, optimal wound healing involves the following processes: (a) rapid hemostasis; (b) appropriate inflammation; (c) mesenchymal cell (MSCs) differentiation, proliferation, and migration to a wound site; (d) suitable angiogenesis (microvascular regeneration); (e) prompt re-epithelialization (re-growth of an epithelial tissue over a wound surface); and (f) proper synthesis, cross-linking, and alignment of collagen to provide strength to a healing tissue.

In recent years, electrical stimulation has been proven to accelerate re-epithelialization and revascularization for promotion of dermal reconstruction. Based on a medical material that has liquid absorbing and moisturizing properties and is attached and loaded with different metal particles on its surface, microcurrent dressings are novel wound dressings with inherent electrical activity. In the presence of moisture and without the need for an external power supply system, the microcurrent dressings can generate a low-level microcurrent on wound contact surfaces to accelerate wound healing. Therefore, how to enhance the stability of the microcurrent dressing and prolong the microcurrent, so as to maintain the antimicrobial function and accelerate wound healing effects, has become an important issue yet to be solved in this field.

### SUMMARY OF THE DISCLOSURE

In response to the above-referenced technical inadequacies, the present disclosure provides an antimicrobial electrochemical fabric and a method for manufacturing the same, so as to effectively increase a contact surface area with wound tissue fluid, and have long-term antimicrobial and wound healing effects.

In order to solve the above-mentioned problems, one of the technical aspects adopted by the present disclosure is to provide a method for manufacturing an antimicrobial electrochemical fabric. The method includes the following steps: providing an electro-spinning polymer solution, in which the electro-spinning polymer solution includes a polymer and a plurality of antimicrobial metal precursors; electro-spinning the electro-spinning polymer solution into a polymer fiber for formation of a sheet structure, in which the plurality of antimicrobial metal precursors are distributed on the polymer fiber; and reducing the plurality of antimicrobial metal precursors into a plurality of antimicrobial metal particles, so as to form the sheet structure into the antimicrobial electrochemical fabric.

In one of the possible or preferred embodiments, the antimicrobial metal particles are silver nanoparticles and zinc nanoparticles.

In one of the possible or preferred embodiments, the polymer fiber is selected from the group consisting of ethyl cellulose, polyethylene glycol, polyethylene terephthalate, polymethyl methacrylate, polystyrene, cellulose ether, chitosan, and sodium alginate.

In one of the possible or preferred embodiments, a diameter of the polymer fiber ranges from 250 nm to 500 nm, and a particle diameter of each of the antimicrobial metal particles ranges from 50 nm to 100 nm.

In order to solve the above-mentioned problems, another one of the technical aspects adopted by the present disclosure is to provide an antimicrobial electrochemical fabric, which is made of a polymer fiber. The polymer fiber includes a plurality of microbatteries and a polymer substrate carrying the plurality of microbatteries. Each of the plurality of microbatteries has at least one pair of electrodes embedded on or within the polymer substrate, and the at least one pair of electrodes includes a first electrode and a second electrode.

In one of the possible or preferred embodiments, the first electrode is a cathode, and the first electrode is made of silver or silver oxide. The second electrode is an anode, and the second electrode is made of zinc.

In one of the possible or preferred embodiments, the first electrode and the second electrode are selected from the group consisting of silver, silver compounds, gold, gold compounds, platinum, platinum compounds, and zinc.

In one of the possible or preferred embodiments, a diameter of the polymer fiber ranges from 250 nm to 500 nm, and a diameter of each of the first electrode and the second electrode ranges from 50 nm to 100 nm.

In one of the possible or preferred embodiments, the plurality of microbatteries are distributed on the polymer fiber and occupy 25% to 40% of a surface area of the polymer fiber.

Therefore, in the antimicrobial electrochemical fabric and the method for manufacturing the same provided by the present disclosure, by virtue of “electro-spinning the electro-spinning polymer solution into a polymer fiber for formation of a sheet structure” and “reducing the plurality of antimicrobial metal precursors into a plurality of antimicrobial metal particles,” the surface area can be increased within a certain unit area, and a micro electric field can be generated, so as to obtain long-term antimicrobial and wound healing effects.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of an antimicrobial electrochemical fabric according to a first embodiment of the present disclosure; and

FIG. 2 is a flowchart of a method for manufacturing the antimicrobial electrochemical fabric according to a second embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

In recent years, for a microcurrent therapy, it is common to externally place a power supply device on an affected part of a wound, so as to generate a microcurrent at the wound (which can promote wound healing). Alternatively, a metallic paint can be applied to a surface of a fabric by way of screen printing, and the fabric can serve as a dressing. However, the external power supply will limit a use environment, and does not allow a patient to move freely. Moreover, the screen-printed microcurrent dressing has a size limitation due to its manufacturing method, and only one side of the dressing has a microcurrent effect. Therefore, in the present disclosure, an antimicrobial electrochemical structure is provided, which can be used for affected parts having different sizes, increase a contact surface area with tissue fluid, provide an entire (stereoscopic) microcurrent electric field, and provide a stable and long-term antimicrobial and pro-healing effect.

### First Embodiment

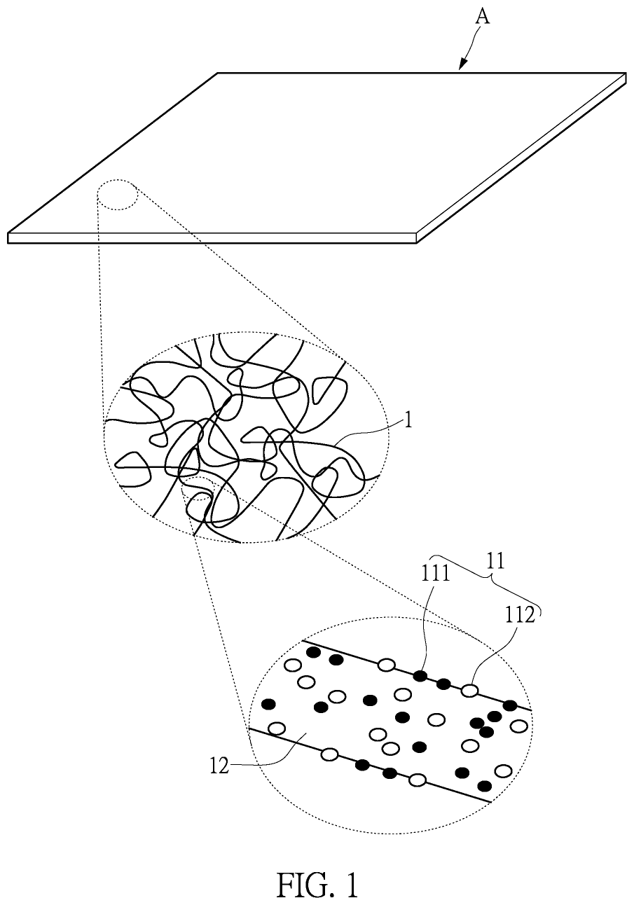

Referring to FIG. 1, a first embodiment of the present disclosure provides an antimicrobial electrochemical fabric A, which is mainly made of a polymer fiber 1. Specifically, the antimicrobial electrochemical fabric A can be formed by stacking, interweaving, or winding one or multiple ones of the polymer fiber 1 in a specific direction or non-specific directions. In this embodiment, the antimicrobial electrochemical fabric A can be formed into a sheet fabric from the polymer fiber 1 in a non-woven manner, and the sheet fabric can have a length and a width ranging from 3 cm to 15 cm. In this embodiment, a thickness of the antimicrobial electrochemical fabric A can be from 0.1 μm to 100 μm, and is preferably from 10 μm to 60 μm. A diameter of the polymer fiber 1 can be from 50 nm to 50,000 nm, is preferably from 1,000 nm to 3,000 nm, and is more preferably from 250 nm to 500 nm. However, the aforementioned details are disclosed for exemplary purposes only, and are not meant to limit the scope of the present disclosure. In practical use, the shape and the size of the antimicrobial electrochemical fabric A can be cut and adjusted according to the size of the wound.

Referring to FIG. 1, the polymer fiber 1 includes a plurality of microbatteries 11 and a polymer substrate 12 carrying the plurality of microbatteries 11. In this embodiment, each of the plurality of microbatteries 11 has at least one pair of electrodes embedded on or within the polymer substrate 12, and the least one pair of electrodes includes a first electrode 111 and a second electrode 112. In this embodiment, the polymer substrate 12 is made of an electrical insulating material. For instance, the material of the polymer substrate 12 can be polyethylene terephthalate (PET), polymethyl methacrylate (PMMA), polystyrene (PS), cellulose ether, chitosan, or sodium alginate. However, the present disclosure is not limited to the above-mentioned examples. Taking mechanical properties and processability into consideration, the material of the polymer substrate 12 is preferably polyethylene terephthalate (PET) having a high crystallinity, polymethyl methacrylate (PMMA) having a low softening temperature, or polystyrene (PS) having a low softening temperature.

In this embodiment, the plurality of microbatteries 11 are distributed on the polymer substrate 12. In particular, the first electrode 111 and the second electrode 112 are different antimicrobial metal particles, and the material of each of the first electrode 111 and the second electrode 112 can be selected from the group consisting of silver, silver compounds, gold, gold compounds, platinum, platinum compounds, and zinc. Specifically, distribution of the plurality of microbatteries 11 takes up about 0.001% to 50% of a surface area of the polymer fiber 1. Preferably, the microbatteries 11 take up 25% to 40% of the surface area of the polymer fiber 1.

For instance, the first electrode 111 can be a cathode, and is silver nano-metal particles or silver oxide nano-metal particles. The second electrode 112 can be an anode, and is zinc nano-metal particles. In this embodiment, the diameter range of the first electrode 111 and the second electrode 112 can be 1 nm to 10,000 nm. Preferably, the first electrode 111 is the silver nano-metal particles having a particle size of from 50 nm to 60 nm, and the second electrode 112 is zinc metal particles having a particle size of from 150 nm to 300 nm. However, the aforementioned details are disclosed for exemplary purposes only, and are not meant to limit the scope of the present disclosure.

In practical use, the antimicrobial electrochemical fabric A can absorb wound tissue fluid as a conductive medium, and through an oxidation-reduction reaction between the first electrode 111 and the second electrode 112, a micro electric field can be generated at the wound without the external power supply, so as to achieve an effect of promoting wound healing. It should be noted that the polymer fiber 1 of the antimicrobial electrochemical fabric A in the present disclosure ranges from a micron level to a nanometer level, and the plurality of microbatteries 11 belong to the nanometer level. Compared with a conventional fabric printing method, the antimicrobial electrochemical fabric A of the present disclosure can expand a three-dimensional space to generate the micro electric field, and have a larger contact surface area with the wound (tissue fluid), thereby achieving long-term antimicrobial and pro-healing effects.

### Second Embodiment

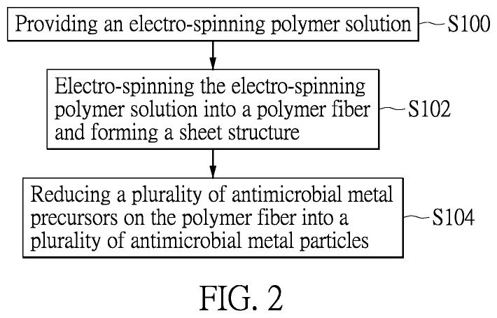

Referring to FIG. 2, which is to be read in conjunction with FIG. 1, a second embodiment of the present disclosure provides a method for manufacturing the antimicrobial electrochemical fabric A. The antimicrobial electrochemical fabric A of the first embodiment of the present disclosure can be obtained through the method of this embodiment. The method of this embodiment includes at least the following main steps: providing an electro-spinning polymer solution (step S100); electro-spinning the electro-spinning polymer solution into a polymer fiber and forming a sheet structure (step S102); and reducing a plurality of antimicrobial metal precursors on the polymer fiber into a plurality of antimicrobial metal particles (step S104). Each step of the method provided by the present disclosure will be described in detail below.

Firstly, in step S100, the electro-spinning polymer solution is provided. The electro-spinning polymer solution can at least include a polymer and the plurality of antimicrobial metal precursors. Furthermore, the polymer is basically an electrical insulating material, and the polymer can be selected from the group consisting of ethyl cellulose, polyethylene glycol (PEG), polyethylene terephthalate (PET), polymethyl methacrylate (PMMA), polystyrene (PS), cellulose ether, chitosan, and sodium alginate. The plurality of antimicrobial metal precursors can be metal salts, metal halides, or metal organic complexes, but the present disclosure is not limited thereto. In practice, the electro-spinning polymer solution can further include an organic solvent, such as methanol or butanone, but the present disclosure is not limited thereto.

For instance, the plurality of antimicrobial metal precursors can be selected from the group consisting of silver precursors, gold precursors, platinum precursors, and zinc precursors. If a metal component of the plurality of antimicrobial metal precursors is gold, the precursor thereof may be exemplified as gold trichloride and tetrachloroauric acid. If the metal component of the plurality of antimicrobial metal precursors is silver, the precursor thereof may be exemplified as silver trifluoroacetate, silver acetate, silver nitrate, silver chloride, and silver iodide. If the metal component of the plurality of antimicrobial metal precursors is platinum, the precursor thereof may be exemplified as sodium hexafluoroplatinate. If the metal component of the plurality of antimicrobial metal precursors is zinc, the precursor thereof may be exemplified as zinc acetate and zinc sulfate. However, these are merely examples and are not to be construed as limiting the scope of the present disclosure. In this embodiment, the polymers are ethyl cellulose and polyethylene glycol, and the plurality of antimicrobial metal precursors are the silver precursors and the zinc precursors. Based on the electro-spinning polymer solution, a content of the silver precursors can range from 0.001 wt % to 20 wt % (preferably from 0.1 wt % to 10 wt %), and a content of the zinc precursors can range from 0.001 wt % to 20 wt % (preferably from 0.1 wt % to 10 wt %). In this embodiment, a solid content of the electro-spinning polymer solution ranges from 3 wt % to 30 wt %, and a viscosity of the electro-spinning polymer solution ranges from 100 cP to 100,000 cP. However, the aforementioned details are disclosed for exemplary purposes only, and are not meant to limit the scope of the present disclosure.

The principle of electro-spinning is to fill the polymer solution into a syringe, and to electrically connect positive and negative electrodes of a high-voltage power supply respectively at a nozzle of the syringe and at a collecting plate spaced apart from the nozzle by a working distance. By externally applying the high-voltage power supply, the polymer solution will be charged after being sprayed from the nozzle, and will form nano-scale or micron-scale fibers towards a collector.

Next, in step S102, the electro-spinning polymer solution is electro-spun into the polymer fiber for formation of the sheet structure. In this embodiment, the conditions of the electro-spinning step may be as follows: a voltage is set between 5 kV and 50 kV, the working distance between the electro-spinning nozzle and the collecting plate is from 5 cm to 50 cm, and an advancing speed of the electro-spinning polymer solution is from 0.5 mL/h to 50 mL/h. It should be noted that, in this step, the sheet structure can be formed during the electro-spinning process, or the sheet structure can be formed by processing (such as pressing and stretching) after the electro-spinning process is completed. A diameter of the polymer fiber formed after step S102 can range from 250 nm to 500 nm. In this embodiment, the diameter of the polymer fiber is 344 nm.

Lastly, in step S104, the plurality of antimicrobial metal precursors on the polymer fiber are reduced into the plurality of antimicrobial metal particles. After the sheet structure of the polymer fiber is formed, the plurality of antimicrobial metal precursors on the polymer fiber are reduced into the plurality of antimicrobial metal particles, so as to become the antimicrobial electrochemical fabric A. In this embodiment, a plasma treatment device can be used to reduce the plurality of antimicrobial metal precursors on the polymer fiber. Adopting a plasma treatment to reduce the plurality of antimicrobial metal precursors allows the plurality of antimicrobial metal particles to be embedded on a surface of the polymer fiber, thereby effectively increasing a contact area. Moreover, since the plurality of antimicrobial metal particles are inlaid on the surface of the polymer fiber, the plurality of antimicrobial metal particles are less likely to fall off Specifically, the conditions of the plasma treatment can be as follows: a plasma wattage ranging from 20 W to 2,000 W, a pressure ranging from 0.1 Torr to 760 Torr, and a plasma treatment time ranging from 1 second to 300 seconds. In this embodiment, the plurality of antimicrobial metals particles obtained after step S104 are silver nano-metal particles (equivalent to the first electrode 111 in the first embodiment) and zinc nano-metal particles (equivalent to the second electrode 112 in the first embodiment). In other embodiments, the plasma treatment device can apply a low pressure, high pressure, or atmospheric plasma treatment, and can use inert gas, air, oxygen, nitrogen, or hydrogen plasma. However, the operating conditions of the above-mentioned plasma treatment can be adjusted according to actual needs, and are not to be construed as limiting the scope of the present disclosure. In some other embodiments, the antimicrobial metal precursor can also be reduced in other ways.

### Antimicrobial Test

The antimicrobial electrochemical fabric (hereinafter referred to as “SZ2”) of the first embodiment is subjected to an antimicrobial test. The antimicrobial electrochemical fabric of the present disclosure is subjected to three types of the antimicrobial test: an AATCC 100-antimicrobial fabric test, a 7-day long-term antimicrobial test, and an anti-biofilm test. The above-mentioned antimicrobial tests all use Staphylococcus aureus as a test strain. The test results are shown in Table 1.

TABLE 1

| | Testing time | Antimicrobial rate |
|---|---|---|
| Test items | (hr) | SZ2 |
| AATCC 100- | 24 | >99.9% |
| Antimicrobial Fabric | | |
| Test | | |
| 7-day Long-term | 168 | >99.99% |
| Antimicrobial Test | | |
| Anti-Biofilm Test | 72 | >99.99% |

The test results show that the antimicrobial electrochemical fabric of the present disclosure passes the AATCC 100-antimicrobial fabric test, the 7-day long-term antimicrobial test, and the anti-biofilm test. The antimicrobial rate of SZ2 in the anti-biofilm test is greater than 99.99%.

In practical use, the antimicrobial electrochemical fabric of the present disclosure can be used as a bioelectric dressing to cover the wound. When a conductive medium (e.g., human tissue fluid, sweat, wound exudate, physiological saline, and water) is present, the microcurrent can be generated through the first electrode and the second electrode on the polymer fiber for simulation of the current naturally generated in the body, thereby promoting wound healing. It should be noted that the antimicrobial electrochemical fabric disclosed in the present disclosure can also disrupt biofilm formation and support cell migration, thereby promoting epithelialization.

### BENEFICIAL EFFECTS OF THE EMBODIMENTS

In conclusion, in the antimicrobial electrochemical fabric and the method for manufacturing the same provided by the present disclosure, by virtue of “electro-spinning the electro-spinning polymer solution into a polymer fiber for formation of a sheet structure” and “reducing the plurality of antimicrobial metal precursors into a plurality of antimicrobial metal particles,” the surface area can be increased within a certain unit area, and the micro electric field can be generated, so as to obtain long-term antimicrobial and wound healing effects.

Furthermore, the antimicrobial metal particles in the antimicrobial electrochemical fabric of the present disclosure can be silver. Compared with silver ions, silver metal particles have a longer antimicrobial effect. When the silver metal particles and the zinc metal particles are used in cooperation with each other, the microcurrent can be further generated in the conductive medium (e.g., cell tissue fluid), so as to promote wound healing.

In addition, since medical dressings are consumables, the costs associated therewith should not be too high. The antimicrobial metal particles in the antimicrobial electrochemical fabric of the present disclosure are nano metal particles, and only take up 25% to 40% of the surface area of the polymer fiber. Through a low content of metal particles, the long-term antimicrobial and wound healing effects can be achieved.

It should be noted that, for patients with diabetes or poorly healed wounds, repeated dressing changes can damage the wound and result in poor recovery of the wound. However, if the dressing is not changed for a long time, a biofilm (e.g., a Pseudomonas aeruginosa infection) is also likely to be formed on the wound, which can lead to wound deterioration. The antimicrobial electrochemical fabric of the present disclosure has 7-day, long-term antimicrobial and anti-biofilm effects. Therefore, the number of times that the dressing needs to be changed can be reduced, and formation of the biofilm can be prevented, thereby effectively helping with the healing of wounds.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for manufacturing an antimicrobial electrochemical fabric, comprising:
(A) providing an electro-spinning polymer solution, wherein the electro-spinning polymer solution includes a polymer and a plurality of antimicrobial metal precursors;
(B) electro-spinning the electro-spinning polymer solution into a polymer fiber for formation of a sheet structure, wherein the plurality of antimicrobial metal precursors are distributed on the polymer fiber; and
(C) reducing the plurality of antimicrobial metal precursors into a plurality of antimicrobial metal particles, so as to form the sheet structure into the antimicrobial electrochemical fabric.

2. The method according to claim 1, wherein the antimicrobial metal particles are silver nanoparticles and zinc nanoparticles.

3. The method according to claim 1, wherein the polymer fiber is selected from the group consisting of ethyl cellulose, polyethylene glycol, polyethylene terephthalate, polymethyl methacrylate, polystyrene, cellulose ether, chitosan, and sodium alginate.

4. The method according to claim 1, wherein a diameter of the polymer fiber ranges from 250 nm to 500 nm, and a particle diameter of each of the antimicrobial metal particles ranges from 50 nm to 100 nm.

5. An antimicrobial electrochemical fabric, characterized in that the antimicrobial electrochemical fabric is made of a polymer fiber, and the polymer fiber includes:
a plurality of microbatteries; and
a polymer substrate carrying the plurality of microbatteries;
wherein each of the plurality of microbatteries has at least one pair of electrodes embedded on or within the polymer substrate, and the at least one pair of electrodes includes a first electrode and a second electrode.

6. The antimicrobial electrochemical fabric according to claim 5, wherein the first electrode is a cathode, and the first electrode is made of silver or silver oxide; wherein the second electrode is an anode, and the second electrode is made of zinc.

7. The antimicrobial electrochemical fabric according to claim 5, wherein the first electrode and the second electrode are selected from the group consisting of silver, silver compounds, gold, gold compounds, platinum, platinum compounds, and zinc.

8. The antimicrobial electrochemical fabric according to claim 5, wherein a diameter of the polymer fiber ranges from 250 nm to 500 nm, and a diameter of each of the first electrode and the second electrode ranges from 50 nm to 100 nm.

9. The antimicrobial electrochemical fabric according to claim 5, wherein the plurality of microbatteries are distributed on the polymer fiber and occupy 25% to 40% of a surface area of the polymer fiber.
